(19) United States
(12) Patent Application Publication
(10) Pub. No.: US 2007/0202082 A1
(43) Pub. Date: Aug. 30, 2007

(54) Promoters for RNA interference

(76) Inventors: Dong-Yan Jin, South Horizons (HK); Elizabeth Yee-Wai Choy, Olympian City (HK); Kin-Hang Kok, Kowloon (HK)

Correspondence Address: COOPER & DUNHAM, LLP, 1185 AVENUE OF THE AMERICAS, NEW YORK, NY, 10036, US

(21) Appl. No.: 11/594,623

(22) Filed: Nov. 7, 2006

Related U.S. Application Data
(60) Provisional application No. 60/735,059, filed on Nov. 9, 2005.

(51) Int. Cl. A61K 48/00; C12N 5/08; C12N 15/86

(52) U.S. Cl. 424/93.2; 435/366; 435/456

(57) ABSTRACT

This invention provides vector systems based on the promoters of Epstein-Barr virus-encoded small RNAs that can be used to express and deliver desired RNA molecules such as small hairpin RNAs in mammalian cells. Such small hairpin RNAs are useful for RNA interference.

### CROSS-REFERENCE TO RELATED APPLICATIONS

This application claims priority of U.S. Provisional Patent Application Ser. No. 60/735,059, filed on Nov. 9, 2005, the entire contents of which is incorporated herein by reference.

### FIELD OF THE INVENTION

This invention relates to vector systems based upon promoters of Epstein-Barr Virus encoded small RNAs that can be used to express small hairpin RNAs useful for RNA interference.

### BACKGROUND OF THE INVENTION

Progress in Human Genome Project promises to revolutionize pharmacology. Whereas in the past, drug discovery relied substantially on finding natural products, often by chance, that can mimic or antagonize the actions of proteins, now we have the opportunities to selectively inhibit the production of proteins. In the past two decades, several types of nucleic acid therapeutics that selectively inhibit protein production have been explored. Particularly, antisense technology has been the subject of great interest and some success has been achieved in the application of antisense oligonucleotides and ribozymes. In addition, emerging evidence indicates that RNA interference is another extremely powerful tool that can be used to block the expression of apparently all genes in a sequence-specific manner (Stevenson, 2004).

RNA interference (RNAi) is an evolutionarily conserved mechanism for post-transcriptional gene silencing, which is mediated by the introduction of double-stranded RNA (dsRNA) triggers and leads ultimately to sequence-specific degradation of the homologous mRNA (Zamore, 2000). This phenomenon was first discovered in Caenorhabditis elegans by injecting long dsRNA (Fire, 1998). However, introduction of dsRNA longer than 30 base pairs Into the mammalian cells induces the interferon response, in which the activation of dsRNA-dependent protein kinase (PKR) and 2′,5′-oligoadenylate synthetase (2′,5′-AS) results in non-specific RNA degradation. To circumvent this pathway, specific gene silencing can be achieved by direct introduction of either chemically synthesized or in vitro transcribed 21-nucleotide long short interfering RNAs (siRNAS) (Elbashir, 2001). Alternatively, short hairpin RNAs (shRNAs) can be expressed from a DNA vector and subsequently processed into functional siRNAs in the cell by Dicer ribonuclease (Paddison, 2002).

Although some RNA polymerase II (Pol II) promoters have been used to express shRNAs in mammalian cells (Xia et al., 2002; Denti et al., 2004), at present shRNAs are more commonly transcribed by mammalian U6 or H1 promoters (Paddison, 2002). The U6 and H1 promoters belong to type III RNA polymerase III (Pol III) promoters that have promoter elements located extragenically. Recent studies have shown that type II Pol III promoters, such as the tRNA promoters having promoter elements located intragenically, can also be used to drive shRNA expression (Kawasaki and Taira, 2003; Boden, 2003) Since the intragenic promoter elements of these promoters are co-transcribed as the 5′ end of the shRNA, the secondary structure formed may confer extra stability to the overall shRNA structure and may help Dicer to assess the shRNA for processing in the initiation step of RNAi (Kawasaki and Taira, 2003).

Animal viruses encode various forms of small RNAs including microRNAs (Pfeffer et al., 2004). While the biological function of most viral small RNAs remains elusive, some of these RNAs, such adenovirus VAI and Epstein-Barr virus-encoded small RNAs (EBERs), are exceedingly abundant in infected cells (Howe and Shu, 1989). The EBERs with a copy number of approximately 107 per cell are by far the most abundant RNAs in EBV-infected cells. EBER1 and EBER2 have 165 and 169 nucleotides, respectively. The EBER promoters are transcribed by Pol III but they are also regulated by transcription factors Sp1 and ATF that bind normally to Pol II promoters. They contain both extragenic and intragenic promoter elements. The extragenic elements include Sp1, ATF and EBER TATA box (ETAB), whereas box A and box B are in the intragenic region (Howe and Shu, 1989). The EBER promoters with these unique features may be useful for driving the expression and delivery of RNAi in mammalian cells.

The patent literature also discusses various promoters. For example, U.S. Pat. No. 6,165,749 (Sagawa) discusses an expression vector using SP6 RNA polymerase. U.S. Pat. No. 6,830,923 (Beug) relates to a genetic unit for inhibiting RNA including the transcription units necessary for transcription by polymerase III. U.S. Pat. No. 5,837,503 (Doglio) relates to a recombinant vector containing a cassette for transcription by RNA polymerase III, wherein a viral gene transcribed by the polymerase has a DNA fragment inserted between or outside boxes A and B, the promoter of the viral gene. In U.S. Patent Application Publication No. 2005/0130184, the patentees discuss compositions for interference RNA including promoters such as the Poly III U6 promoter. Further, U.S. Patent Application Publication No. 2003/0144239 (Agami), discusses a polynucleotide including RNA polymerase III promoter, a region encoding an siRNA and a transcriptional termination element comprising five consecutive thymidine residues. The foregoing patents and applications are incorporated by reference herein.

There exists a continued need for improved and different promoters involved in gene silencing, RNAi and nucleic acid therapeutics.

### SUMMARY OF THE INVENTION

It is an object of invention to provide compositions and methods for using DNA vectors based on the EBER promoters to express and deliver target RNAs in mammalian cells.

It is a further object of the invention to provide compositions of vector systems for expression and delivery of desired RNAs into a host cell, comprising an expression cassette, which comprises EBER1 or EBER2 promoter operably linked to a nucleic acid sequence.

This invention provides vector systems, wherein the expression cassette comprises an EBER1 or EBER2 promoter operably linked to a nucleic acid sequence encoding a small interfering RNA (siRNA), wherein the siRNA comprises a first region and a second region, wherein at least a portion of the first region is complementary to the second region so that a double stranded RNA comprising about 18 to about 25 nucleotides is formed.

This invention further provides compositions of vector systems driven by EBER1 or EBER2 promoter, wherein the siRNA is a small hairpin RNA (shRNA). This invention further provides compositions of vector systems driven by EBER1 or EBER2 promoter, wherein at least a portion of the siRNA is complementary to a target RNA, wherein the portion is about 15 to about 19 nucleotides in length.

This invention additionally provides methods for inhibiting the function of a target RNA, which comprises transfecting mammalian and human cells with any of the vector systems described above.

The vectors are a useful tool for the delivery of gene silencing agents in mammalian cells and for developing nucleic acid therapeutics.

### BRIEF DESCRIPTION OF THE FIGURES

FIG. 1 is a schematic diagram of shRNA expression vectors. All vectors can drive the expression of shRNA (sense-loop-antisense: [symbol] □ [symbol] ), which is terminated by a stretch of six thymidines. Three restriction sites (XbaI, ClaI and XhoI) were inserted upstream of the shRNA sequence to facilitate subcloning. pEBER1-shRNA and pEBER2-shRNA have incorporated all the extragenic (Sp₁, ATF and ETAB) and intragenic (box A and box B) promoter elements of the EBER promoters. The U6+1 and U6+27 promoters are more efficient than just simply U6 promoter. DSE: distal sequence element. PSE: proximal sequence element.

FIG. 2 shows gene silencing activity of shRNAs expressed from pEBER1-shRNA and pEBER2-shRNA vectors. (A) Silencing of Fluc expression. HeLa cells were transfected with luciferase reporter plasmids pLuc alone or pLuc plus the indicated expression vectors. pEBER1-shFluc and pEBER2-shFluc were driven by EBER promoters and they expressed an shRNA targeting Fluc (shFluc). pEBER1-T6 and pEBER2-T6 were empty vectors containing EBER promoters and transcription termination signal T₆. pSHAG-Ff1 was a control plasmid previously known to express shFluc efficiently. The relative Fluc activity was obtained by normalizing Fluc readouts with those of Rluc. The relative Fluc activity recovered from cells receiving pLuc alone was set as 100%. Results represent the average of triplicate experiments and the error bars indicate standard deviation. (B) Inhibition of Rluc expression. pEBER1-shRluc and pEBER2-shRluc were expression vectors for shRNA targeting Rluc (shRluc) driven by EBER promoters. pRL1776 was a control plasmid known to express shRluc effectively. The relative Rluc activity was obtained by normalizing Rluc readouts with those of Fluc. The relative Rluc activity recovered from cells transfected with pLuc alone was set as 100%. Results represent the average of triplicate experiments and the error bars indicate standard deviation. (C) Specificity of RNAi effect. Cells were transfected with the Fluc and lacZ reporter plasmids alone (reporters alone) or the reporter plasmids plus the indicated expression vectors. The relative β-galactosidase activity was obtained by normalizing β-galactosidase activity readouts with those of Fluc. The relative β-galactosidase activity of cells having reporters alone was set as 100%. Results represent the average of triplicate experiments and the error bars indicate standard deviation.

FIG. 3 Comparison of shRNA expression vectors. Three different cell lines (HeLa, HEK293 and CNE2) were transfected with reporter plasmids pLuc and the indicated shRNA expression vectors (see FIG. 1 for reference). Results were normalized to Fluc activity and the average of triplicate experiments were shown. The error bars indicate standard deviation. The relative Rluc activity recovered from pLuc was set as 100%.

FIG. 4. Analysis of shRNAs and siRNAs in cells transfected with pEBER1-shRluc and pEBER2-shRluc vectors. (A) Northern blotting. Total cellular RNA was extracted from HeLa cells transfected with the indicated expression plasmids. (B) RPA. RNA was extracted and enriched from HEK293 cells transfected with the indicated expression plasmids. Solution hybridization and RNase treatment were carried out, followed by analysis of protected fragments on a 15% urea gel. Non-specific hybridization signal was marked with an asterisk.

FIG. 5. Quantitative PCR analysis of mRNA degradation induced by shRNAs expressed from pEBER1-shRluc and pEBER2-shRluc vectors. Each bar represents the average of triplicate experiments and standard deviation was also plotted. Normalization with the Fluc mRNA level was carded out to obtain the relative Rluc mRNA level. The relative Rluc mRNA level of pLuc-transfected cells was set as 100%. nt: nucleotides.

### DETAILED DESCRIPTION OF THE PREFERRED EMBODIMENTS

RNA interference (RNAi) is an evolutionarily conserved mechanism for post-transcriptional gene silencing, which is mediated by the introduction of double-stranded RNA (dsRNA) triggers (1,2) and leads ultimately to sequence-specific degradation of the homologous mRNA (3,4). This phenomenon was first discovered in Caenorhabditis elegans by injecting long dsRNA (1). However, introduction of dsRNA longer than 30 base pairs into the mammalian cells induces the interferon response, in which the activation of dsRNA-dependent protein kinase (PKR) and 2′,5′-oligoadenylate synthetase (2′,5′-AS) results in non-specific RNA degradation (5-7). To circumvent this pathway, specific gene silencing can be achieved by direct introduction of either chemically synthesized (8) or in vitro transcribed 21-nucleotide long short interfering RNAs (siRNAs) (9,10). Alternatively, short hairpin RNAs (shRNAs) can be expressed from a DNA vector and subsequently processed into functional siRNAs in the cell by Dicer ribonuclease (9,11-18).

Although some RNA polymerase II (Pol II) promoters have been, used to express shRNAs in mammalian cells (19,20), at present shRNAs are more commonly transcribed by mammalian U6 or H1 promoters (9,11-17). The U6 and H1 promoters belong to type III RNA polymerase III (Pol III) promoters that have promoter elements located extragenically (21). Recent studies have shown that type II Pol III promoters, such as the tRNA promoters having promoter elements located intragenically, can also be used to drive shRNA expression (22,23). Since the intragenic promoter elements of these promoters are co-transcribed as the 5′ end of the shRNA, the secondary structure formed may confer extra stability to the overall shRNA structure and may help Dicer to assess the shRNA for processing in the initiation step of RNAi (22).

Animal viruses encode various forms of small RNAs including microRNAs (24). While the biological function of most viral small RNAs remains elusive, some of these RNAs, such adenovirus VAI and Epstein-Barr virus-encoded small RNAs (EBERs), are exceedingly abundant in infected cells (25-27). In light of this, we sought to investigate whether promoters of viral small RNAs such as EBERs can be used to drive the expression of shRNAs in cultured human cells.

The EBERs with a copy number of approximately 10⁷ per cell are by far the most abundant RNAs in EBV-infected cells (26). They are not essential for viral infection and their precise function is poorly defined (27). EBER1 and EBER2 have 165 and 169 nucleotides, respectively. The EBER promoters are transcribed by Pol III but they are also regulated by transcription factors Sp1 and ATF that bind normally to Pol II promoters (26). They contain both extragenic and intragenic promoter elements. The extragenic elements include Sp1, ATF and EBER TATA box (ETAB), whereas box A and box B are in the intragenic region (26-28). The activity of the EBER promoters is dictated by these unique features.

In this study we designed and constructed a new set of DNA vectors driven by the EBER promoters. We showed that these EBER promoters were able to drive the transcription of shRNAs efficiently. In addition, the siRNAs processed from the expressed shRNAs specifically inhibited the expression of luciferase mRNA and protein in various types of mammalian cells to the same degree as those produced by the commonly used RNAi vectors driven by H1 or U6 promoter. Thus, we provided the proof-of-principle that the EBER promoters can be used to drive intracellular expression of shRNAs for induction of RNAi. These vectors based on the EBER promoters are a useful tool for the delivery of gene silencing agents in mammalian cells and for developing nucleic acid therapeutics.

Materials and Methods

Construction of shRNA Expression Vectors

PCR and TA cloning were employed for the construction of shRNA expression vectors. The shRNA expression cassettes containing the promoters (i.e. promoter-sense-loop-antisense-termination signal T₆) were amplified by PCR. The EBER promoters were derived from EBV genomic DNA purified from B95-8 cells (a gift from George Tsao, The University of Hong Kong). Human H1 promoter and different versions of human U6 promoters (U6+1 and U6+27) were PCR-subcloned from plasmids pSUPER (a gift from Reuven Agami, The Netherlands Cancer Institute; Ref. 11) and pGEM-U6 (a gift from Greg Hannon, Cold Spring Harbor Laboratory). Human tRNA^{Val} promoter was PCR-amplified from genomic DNA of HeLa cells.

The shRNA against firefly luciferase (shFluc) was derived from plasmid pSHAG-Ff1 (a gift from Greg Hannon; Ref. 9). The shRNA against Renilla luciferase (shRluc) was targeted to 1776 to 1803 nucleotides of Rluc mRNA. The primers used were as follows: EBER1-shFluc, 5′-GGA AAT GAG GGT TAG CAT AGG C-3′ (forward) and 6′-AAA AAA ATC AGG TGG CTC CCG CTG AAT TGG AAT CCC AAG CTT CGG ATT CCA ACT CAG CGA GAG CCA CCC GAT CTC GAG ATC GAT TCT AGA AGA CAA CCA CAG ACA CCG TC-3′ (reverse); EBER2-shFluc, 5′-GCT TAA CGT TGC ATC CCA GAA G-3′ (forward) and 5′-AAA AAA ATC AGO TGG CTC CCG CTG AAT TGG AAT CCC AAG CTT CGG ATT CCA ACT CAG CGA GAG CCA CCC GAT CTC GAG ATC GAT TCT AGA CTG ACT TOC AAA TGC TCT AGG C-3′ (reverse); EBER1-shRluc, 5′-GGA AAT GAG GGT TAG CAT AGG C-3′ (forward) and 5′-AAA AAA GTT AG AAT TAT AAT GOT TAT CTA CGT GCC AAG CTT COC ACA TAG ATA AGC ACT ATA ATC CCT AAC CTC GAG ATC GAT TCT AGA AGA CAA CCA CAG ACA CCG TC-3′ (reverse); EBER2-shRluc, 5′-GCT TAA CGT TGC ATC CCA GAA G-3′ (forward) and 5′-AAA AAA GTT AGG AAT TAT AAT GCT TAT CTA CGT GCC AAG CTT CGC ACA TAG ATA AGC ACT ATA ATC CCT AAC CTC GAG ATC GAT TCT AGA CTG ACT TGC AAA TGC TCT AGG C-3′ (reverse); tRNA(Val)-shRluc, 5′-TTG ATC CCG AAA,GAT GTC CAG CG-3′ (forward) and 5′-AAA AAA GTT AGG AAT TAT AAT GCT TAT CTA CGT GCC AAG CTT CGC ACA TAG ATA AGC ACT ATA ATC CCT AAC CTC GAG ATC GAT TCT AGA TTT CCG CCC GGT TTC GAA C-3′ (reverse); U6+1-shRluc, 5′-ATT TAG GTG ACA CTA TAG-3′ (forward) and 5′-AAA AAA GTT AGG AAT TAT AAT OCT TAT CTA CGT GCC AAG CTT COC ACA TAG ATA AGC ACT ATA ATC CCT AAC CTC GAG ATC GAT TCT AGA CGG TGT TTC GTC CTT TCC AC-3′ (reverse); U6+27-shRluc, 5′-ATT TAG GTG ACA CTA TAG-3′ (forward) and 5′-AAA AAA GTT AGG AAT TAT AAT GCT TAT CTA CGT GCC AAG CTT CGC ACA TAG ATA AGC ACT ATA ATC CCT AAC CTC GAG ATC GAT TCT AGA TAG TAT ATG TGC TGC CGA AGC GAG CAC GGT GTT TCG TCC T-3′ (reverse); H1-shRluc, 5′-TAA TAC GAC TCA CTA TAG GG-3′ (forward) and 5′-AAA AAA GTT AGG AAT TAT AAT GCT TAT CTA CGT GCC AAG CTT CGC ACA TAG ATA AGC ACT ATA ATC CCT AAC TCT AGA CTC GAG ATC GAT GGG AAA GAG TGG TCT CAT ACA GAA CTT ATA AGA TTC CC-3′ (reverse). The resulting products were then separately cloned into pGEM®-T easy vector (Promega).

Cell Line and Transient Transfection

HeLa and HEK293 cells were cultured in Dulbecco's modified Eagle's medium supplemented with 10% fetal bovine serum (FBS). CNE2 cells were grown in RPMI1640 medium supplemented with 10% FBS. Cells were transfected at 50% confluence in 12-well plate or 10 cm dish for 48 hours using GeneJuice® transfection reagent (Novagen). Plasmids expressing Fluc under the control of SV40 promoter (pGL3-Control) or Rluc driven by the CMV enhancer and early promoter (pRL-CMV) were from Promega.

To knockdown the expression of Fluc, 0.3 μg of pGL3-control, 0.3 μg of pRL-SV40 and 1 μg of shRNA expression vector were co-transfected into cells in each well of the 12-well plates. To target Rluc, these plasmids were co-transfected in a ratio of 1:1:2. As such, cells in each well of the plates received 0.3 μg of pGL3-control, 0.3 μg of pRL-SV40 and 0.6 μg of shRNA expression vector. In testing the specificity of the knockdown mediated by shRluc, pRL-CMV was replaced by pcDNA3.1N5-His/lacZ (Invitrogen) in the transfection.

Dual Luciferase Reporter Assay

Luciferase assays were performed as described (29). Briefly, after rinsing with 1PBS once, the harvested cells were lysed with 150 μl of passive lysis buffer (Promega) at room temperature for 15 min. Subsequently, 5 or 10 μl of the cell lysate was added to an opaque 96-well plate for measurement of luciferase activity using the Dual-Luciferase® reporter assay system (Promega). The readouts of luciferase activity were taken in an LB 96V microplate luminometer (EG&G).

Luminescent β-Galactosidase Reporter Assay

Cells were harvested and lysed with passive lysis buffer. Measurement of β-galactosidase activity was performed using the luminescent β-galactosidase detection kit II (Clontech). The readouts of β-galactosidase activity were taken in an LB 96V microplate luminometer (EG&G).

Northern Blot

Total RNA was harvested 48 h after transfection using Trizol reagent (Invitrogen) 20 μm of total RNA was then separated by electrophoresis on a 12% polyacrylamide/8M urea gel and electroblotted onto a Zeta-Probe GT membrane (Bio-Rad) for 1.5 h at 30 V. RNA was immobilized by UV crosslinking. Hybridization was carried out at 42° C. using Ultrahyb-Oligo hybridization buffer (Ambion) and a ³²P-labeled Rluc sense DNA oligonucleotide. Membrane was washed twice for 20 min each at 42° C. with 2SSC and 0.2% SDS. Film was exposed overnight with intensifying screen.

RNase Protection Assay (RPA)

Small RNAs (around 200 nucleotides or smaller) were extracted and enriched from transfected HEK93 in 10 cm dish using mirVana™ miRNA isolation kit (Ambion). A ³²P-labeled Rluc sense RNA probe was made using the mirVana™ miRNA probe construction kit (Ambion). Solution hybridization was carried out overnight at 42° C. with 10 μg of the enriched RNA and the gel-purified probe. After 24 h of hybridization, RNase treatment was performed for 1 h with 40 μg/ml RNase A (USB) and 2 U of RNase T1 (Ambion). RNase was then inactivated with 5 μl of proteinase K (10 mg/ml) in 10 μl of 20% SDS. The protected fragments were analyzed by polyacrylamide gel electrophoresis with 15% urea. Results were visualized using a phosphor imager (Molecular Dynamics).

Quantitative RT-PCR

Total cellular RNA was extracted using Trizol and proteinase K. Briefly, the cells were incubated with Trizol for 3 min at room temperature. The lysed samples were then treated with 10 μl of preheated proteinase K (10 mg/ml) at 55° C. for 30 min. Total cellular RNA was extracted using the protocol provided by Invitrogen. After RNA extraction, 2 μg of the total cellular RNA was treated with 2 U of DNase (Ambion) at 37° C. for 15 min. DNase was heat-inactivated at 65° C. for 20 min. The synthesis of cDNA with oligo(dT)₂₀ primer was then performed using the ThermoScript™ RT-PCR system (Invitrogen).

Subsequently, 2 μl of the synthesized cDNA was added to the quantitative PCR reaction mix containing 12.5 μl of 2× Brilliant® SYBR® Green QPCR master mix (Stratagene), 2.5 μl of forward primer (1 μM), 2.5 μl of reverse primer (1 μM) and 5.5 μl of water. Primers used to quantitate the Rluc mRNA were 5′-ACG CTG AAA GTG TAG TAG A-3′ (forward) and 5′-AGA ATC CTG GGT CCG A-3′ (reverse). For RNA normalization, quantitation of the Fluc mRNA was performed using 5′-TCT ATC CGC TGG AAG ATG-3′ (forward) and 5′-ACT GTT GAG CAA TTC ACG-3′ (reverse). Quantitative PCR was carried out in Mx3000P™ real-time PCR system (Stratagene) using the following thermal cycling profile: 1 cycle of 95° C. for 10 min, 40 cycles of amplification (95° C. for 30 sec, 55° C. for 1 min and 72° C. for 30 sec), followed by 41 cycles of incubation where the temperature increased by 1° C. per cycle beginning at 55° C. and ending at 95° C. For each experimental setup, triplicate measurements were carried out and analyzed using the software (version 1.20c) provided with the Mx3000P™ system.

Results

Construction of shRNA Expression Vectors Driven by EBER Promoters

The EBER promoters are the strongest viral promoters transcribed by Pol III (26). Similar to tRNA promoters, the EBER promoters contain intragenic elements that are co-transcribed into RNA. The resulting highly-structured transcripts could be more resistant to non-specific degradation (22). In addition, the Sp1- and ATF-binding elements in the EBER promoters may further enhance transcription (27). In light of all these unique features of the EBER promoters, we asked whether they could be utilized to drive the expression of shRNAs in mammalian cells. As a first step, we set out to construct shRNA expression vectors based on the EBER promoters.

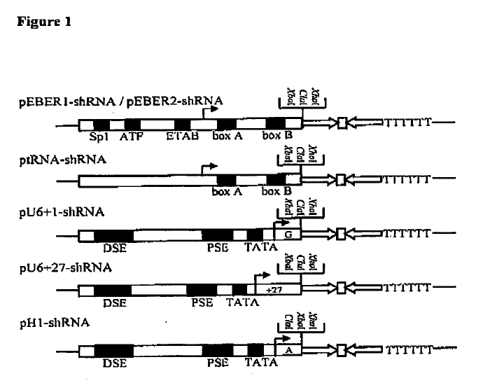

Using PCR cloning, we were able to assemble two shRNA expression vectors pEBER1-shRNA and pEBER2-shRNA controlled by EBER1 and EBER2 promoters, respectively (FIG. 1). These vectors have incorporated all the EBER extragenic and intragenic promoter elements including Sp1 element, ATF motif, ETAB, box A and box B. shRNAs are to be placed downstream the promoter through multiple cloning sites. The transcription of 29-mer shRNA is terminated with a stretch of six thymidines. The shRNA sequence has an 8-nucleotide loop sequence with a HindIII restriction site situated between the sense and antisense strands of the siRNA sequence. For comparison, we also constructed a series of shRNA expression vectors under the control of human tRNA^{Val}, U6 and H1 promoters (FIG. 1). Two versions of modified U6 promoters (U6+1 and U6+27) were designed. These U6+1 and U6+27 promoters have previously been shown to express shRNAs more efficiently than the original U6 promoter (23). Particularly, transcripts from the U6+1 promoter have an additional guanosine at the 5′ end, which facilitates transcription (16). Transcripts from the U6+27 promoter contain a 27-nucleotide sequence of the endogenous U6 RNA, which confers structural stability (14).

Expression of shRNAs Driven by EBER Promoters

Next we investigated whether the expression vectors driven by the EBER promoters might be used to produce shRNAs in cultured human cells. For this purpose, we chose an shRNA directed against Fluc (shFluc), whose effectiveness in knocking down the expression of its target gene has been well documented (9). The shFluc sequence was subcloned into pEBER1-shRNA and pEBER2-shRNA vectors. These vectors were then co-transfected into HeLa cells together with Fluc and Rluc reporter plasmids. The transfected cells were assayed for Fluc and Rluc activities. In this dual luciferase assay, we used pSHAG-Ff1 plasmid, which has been shown to express shFluc efficiently in cultured cells (9), as a positive control.

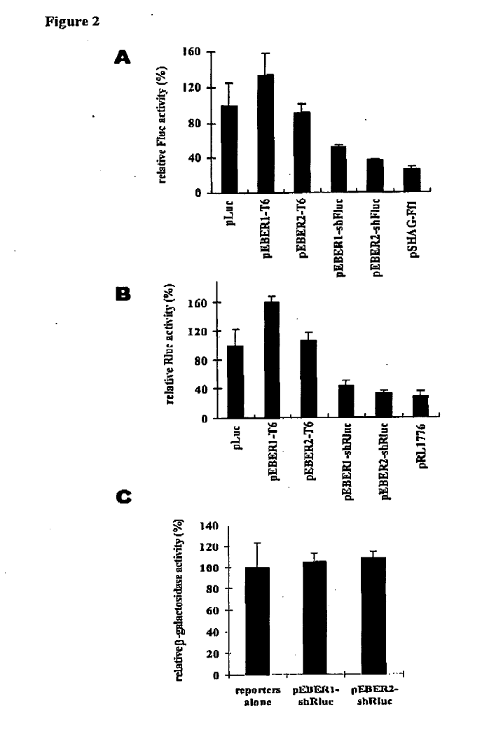

We observed that Fluc activity was significantly reduced in cells transfected with either pEBER1-shFluc or pEBER2-shFlu (FIG. 2A). As such, a 70% inhibition of Fluc activity was achieved in pEBER2-shFluc-transfected cells. A similar reduction was also seen in cells carrying pSHAG-Ff1. In contrast, cells having empty vector pEBER1-T6 or pEBER2-T6 were able to express Fluc to the same level as cells transfected with reporter plasmids alone (pLuc). Notably, the readouts of Rluc activity In cells carrying pEBER1-shFluc, pEBER2-shFluc or pSHAG-Ff1 did not drop significantly (data not shown), indicating that the reduction in Fluc activity was rather specific. These results suggest that EBER promoters can be used to express shRNAs.

To verify this, we employed the EBER promoters to express another shRNA targeting Rluc (shRluc) but not Fluc. In this case, significant knockdown of Rluc activity was observed in HeLa cells transfected with pEBER1-shRluc or pEBER2-shRluc, when compared to cells carrying plasmid pRL1776, a positive control for expression of shRluc. The inhibition of Rluc activity attributed to plasmid pEBER2-shRluc was around 70% (FIG. 2B). This inhibition was not seen in cells carrying reporter plasmids alone (pLuc) or an empty vector (pEBER1-T6 or pEBER2-T6). In addition, the original readouts of Fluc activity in cells receiving pEBER1-shRluc, pEBER2-shRluc or pRL1776 were comparable to those in cells carrying pLuc reporter plasmids alone (data not shown). Consistent with this, the β-galactosidase activity in cells transfected with pEBER1-shRluc and pEBER2-shRluc did not decrease when compared with cells bearing Rluc and lacZ reporters alone (FIG. 2C), further demonstrating the specificity of RNAi effect. These results indicated that the shRNA expression vectors driven by the EBER promoters did not induce non-specific gene silencing in mammalian cells. Thus, shRNAs expressed from the EBER promoters specifically and significantly inhibited the expression of target genes.

Comparison of EBER Promoters with Other Pol III Promoters for shRNA Expression

Above we showed that EBER promoters were able to drive the expression of shRNAs in HeLa cells (FIG. 2). To further assess their ability to express shRNAs, we compared them with other human Pol III promoters commonly used in shRNA expression. All of these promoters (i.e. EBER1, EBER2, tRNA^{Val}. U6+1, U6+27 and H1 promoters) were Inserted into the same vector backbone (FIG. 1). These shRNA expression vectors were then compared for shRNA-mediated gene silencing activities in three different cell lines.

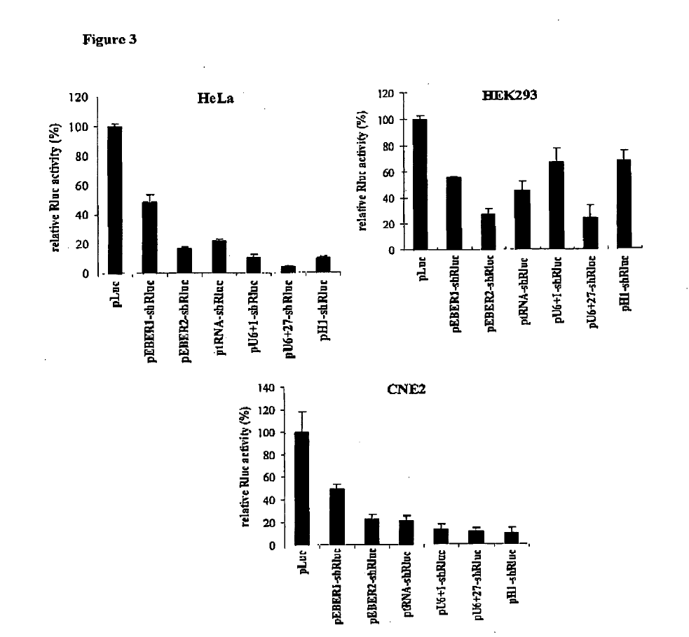

We performed another series of dual luciferase assays with HeLa (human epithelial cervical), HEK293 (human embryonic kidney epithelial) and CNE2 (human nasopharyngeal) cells transfected Individually with the expression vectors (FIG. 3). While both EBER1 and EBER2 promoters were able to drive shRNA expression, the gene silencing activity mediated by shRluc transcribed from EBER2 promoter was consistently higher than that attributed to the same shRNA expressed from EBER1 promoter (FIG. 2 and FIG. 3). Judging from the gene silencing activities, the expression of shRluc from EBER2 promoter was at least as effective as that from the other commonly used Pol III promoters including tRNA^{Val}, U6+1, U6+27 and H1 promoters. pEBER2-shRluc led to a significant knockdown of Rluc activity up to 80%. It is noteworthy that pU6+27-shRluc was able to deliver the greatest gene silencing effect most of the time. This trend of gene silencing was also observed in other human cancer cell lines including nasopharyngeal carcinoma HNE1 cells (data not shown). Generally, the gene silencing activity induced by shRluc expressed from different vectors was cell type-independent (FIG. 3).

Analysis of shRNA, siRNA and mRNA Target in Cells Carrying EBER Promoter-Based Expression Vectors

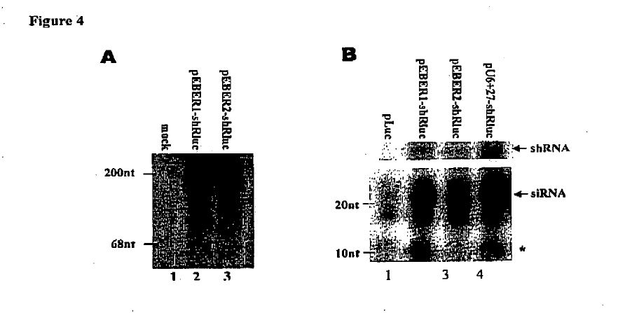

Results from dual luciferase assays support the notion that the EBER promoters can be used to drive the expression of shRNAs as effectively as the commonly used H1, U6 and tRNA^{Val} promoters (FIG. 2 and FIG. 3). However, while reporter assays are sensitive, they only indirectly reflect the gene silencing activities of shRNAs. To verify that specific gene silencing observed In the analysis of luciferase activity is indeed mediated by the shRNA expressed, Northern blotting was performed using the total RNA extracted from cells that had previously shown to have the RNAi effect targeted to Rluc. As expected, ample amount of shRluc of the correct size (194 nucleotides for EBER1-shRluc and 209 nucleotides for EBER2-shRluc) was detected in cells transfected with pEBER1-shRluc or pEBER2-shRluc (FIG. 4A, lanes 2 and 3 compared to lane 1).

shRNAs expressed in cells are thought to be processed by Dicer ribonuclease to generate siRNAs, which induce RNAi effect (30). Earlier in this study we demonstrated the expression of shRNA from the EBER promoters (FIG. 4A) and the gene silencing activity of the shRNA expressed (FIG. 2 and FIG. 3). However, it remained to be determined whether the gene silencing effect might be attributed to siRNAs generated from the shRNA transcribed from the EBER promoters. To confirm the processing of shRNA transcripts of the EBER promoters, we performed an RPA assay using the enriched RNA isolated from HEK293 cells transfected with shRluc expression vectors driven by the EBER and U6+27 promoters (FIG. 4B). We noted that the steady-state amount of shRNAs in cells transfected with pEBER1-shRluc or pEBER2-shRluc was the same as that in cells carrying pU6+27-shRluc (FIG. 4B, upper panel, lanes 2 and 3 compared to lane 4). Importantly, siRNAs specifically hybridized to the Rluc sense probe were found in cells transfected with pEBER1-shRluc or pEBER2-shRluc (FIG. 43, lower panel, lanes 2 and 3). siRNAs were also detected In cells carrying pU6+27-shRluc (FIG. 4B, lower panel, lane 4), but not in cells receiving pLuc reporter plasmids alone (lane 1). Thus, effector siRNAs were generated from shRluc transcribed from the EBER promoters.

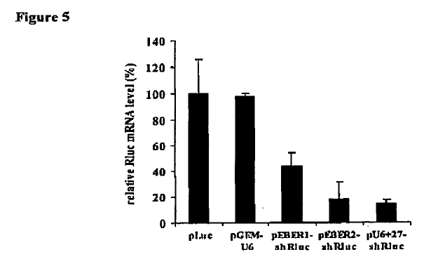

The detection of siRNAs in cells carrying shRNA expression vectors driven by the EBER promoters prompted us to ask whether the gene silencing effect we observed (FIG. 2 and FIG. 3) was due to siRNA-induced degradation of the target mRNA. To address this issue, we performed quantitative RT-PCR using total RNA extracted from pEBER1-shRluc- and pEBER2-shRluc-transfected HEK293 cells (FIG. 5). Another siRNA expression plasmid pU6+27-shRluc was chosen for comparison due to its greatest silencing effect. Additionally, an empty siRNA expression vector pGEM-U6 was used as a negative control in this experiment. We noted that the relative Rluc mRNA level did decrease in pEBER1-shRluc-transfected cells. The Rluc mRNA level dropped to a more significant degree in cells having pEBER2-shRluc. Around 85% of the targeted Rluc mRNA was cleaved and this ratio was comparable to that observed in pU6+27-shRluc-transfected cells. Hence, shRNAs transcribed from the EBER promoters were efficiently processed into siRNAs that induce sequence-specific mRNA degradation.

In this study, we provide the first evidence that the EBER promoters from EBV can be used to express shRNAs in cultured mammalian cells. shRNAs were abundantly transcribed from the EBER promoters (FIG. 4A) and were subsequently processed presumably by endogenous Dicer nuclease into siRNAs (FIG. 4B). The effector siRNAs were able to induce sequence-specific degradation of target mRNA (FIG. 5) leading ultimately to the knockdown of protein expression as Indicated in the reduction of protein activity (FIG. 2). The expression of shRNAs by the EBER promoters was cell type-independent and was as effective as that from other commonly used Pol III promoters (FIG. 3).

Ever since the discovery that 21-nucleotide siRNAs introduced into mammalian cells effectively induce RNAi without provoking the interferon response (8), various strategies have been used to achieve the greatest and the most stable gene silencing effect within the cells (30). One breakthrough is the use of DNA vectors to express shRNAs (9,11-18). The shRNA has a fold-back stem-loop structure, such that it can ultimately be processed by Dicer into functional siRNA (30). Although some Pol II promoters including CMV and U1 promoters, have been shown to be able to deliver shRNAs into the cells (19, 20), until now one common design of RNAi experiments is still to express shRNAs from a DNA vector driven by Pol III promoters such as U6, H1 and tRNA promoters (9,11-18,22,23). Our work has added one important group of viral Pol III promoters to the list of promoters that can drive shRNA expression. Our findings establish the concept that shRNAs transcribed from Pol III viral promoters are effectively processed by Dicer into functional siRNAs.

EBERs are most abundantly found in the infected cells, having 10 copies per cell (26). The EBER promoters are among the strongest promoters ever known. The shRNAs transcribed from the EBER promoters were highly abundant in cells (FIG. 4A). The degradation of mRNA induced by these shRNAs was also remarkable (FIG. 5). However, the gene silencing activity of the shRNA expressed from the EBER promoters was lower than that of the same shRNA transcribed from the U6+27 promoter (FIG. 3). For an unknown reason, shRNAs expressed from the EBER2 promoter were able to knockdown target gene expression more efficiently than those transcribed from EBER1 promoter (FIGS. 2 and 3). Additional experiments are required to elucidate whether the difference in gene silencing activity could be explained by the levels, localization or stability of shRNAs expressed in the cells. Based on the new information obtained from these experiments, the existing pEBER1-shRNA and pEBER2-shRNA vectors can be further modified and improved One recent study suggests that efficient transcription from the EBER2 promoter depends on the secondary structure of the RNA (31). Thus, further modification of the shRNA sequence might enhance its transcription from the EBER promoters.

The EBER promoters have other unique features that can be utilized in the construction of shRNA expression vectors. Particularly, they have both extragenic (Sp1, ATF and ETAB) and intragenic promoter elements (box A, and box B) (26-28).

All these elements are important for the transcriptional activity of the EBER promoters (26-28) and they were all incorporated into the existing pEBER1-shRNA and pEBER2-shRNA vectors (FIG. 1). In this regard, the extragenic promoter elements may provide an opportunity for further improvement and development of the existing plasmids into an enhanced and inducible shRNA expression vector. On the other hand, the intragenic promoter elements co-transcribed with the shRNA sequence provide additional stability to the shRNA and may also improve the accessibility of shRNA to Dicer, as in the case of U6+27 and tRNA promoters (23).

The EBERs localize to the nucleus (32). It will be of great interest to determine the subcellular localization of shRNAs that are transcribed from the EBER promoters and that contain a 5′ sequence derived from the EBERs. In this regard, some shRNAs expressed from U6 promoter have been found mainly in the nucleus (22). Interestingly, while RNAi mediated by mRNA degradation occurs in the cytoplasm (33), one recent study has revealed that siRNAs can also induce specific RNA degradation in the nucleus (34). In addition, nuclear siRNAs are capable of inducing specific gene silencing through promoter hypermethylation (35,36). Hence, shRNAs and siRNAs targeted to the cytoplasm and the nucleus can be used to knockdown the levels of different RNAs specifically through at least three different mechanisms.

### REFERENCES

The following references are incorporated by reference herein:
1. Fire, A., Xu, S., Montgomery, M. K., Kostas, S. A., Driver, S. E. and Mello, C. C. (1998) Potent and specific genetic interference by double-stranded RNA in Caenorhabditis elegans. Nature, 391, 806-811.
2. Montgomery, M. K., Xu, S. and Fire, A. (1998) RNA as a target of double-stranded RNA-mediated genetic interference in Caenorhabditis elegans. Proc. Natl. Acad. Sci. USA, 95, 15502-15507.
3. Zamore, P. D., Tuschl, T., Sharp, P. A. and Bartel, D. P. (2000) RNAi: double stranded RNA directs the ATP-dependent cleavage of mRNA at 21 to 23 nucleotide intervals. Cell, 101, 25-33.
4. Elbashir, S. M., Martinez, J., Patkaniowska, A., Lendeckel, W. and Tuschl, T. (2001) Functional anatomy of siRNAs for mediating efficient RNA in Drosophila melanogaster embryo lysate. EMBO J., 20, 6877-6888.
5. Samuel, C. E. (1993) The eIF-2α protein kinases, regulators of translation in eukaryotes from yeasts to humans. J. Biol. Chem., 268, 7603-7606.
6. Der, S. D., Yang, Y. L., Weissmann, C. and Williams, B. R. (1997) A double-stranded RNA-activated protein kinase-dependent pathway mediating stress-induced apoptosis. Proc. Nat. Acad. Sci. USA, 94, 3279-3283.
7. Ghosh, S. K., Kusari, J., Bandyopadhyay, S. K., Samanta, H., Kumar, R. and Sen, G. C. (1991) Cloning, sequencing, and expression of two murine 2′-5′-oligoadenylate synthetases: Structure-function relationships. J. Biol. Chem., 266, 15293-15299.
8. Elbashir, S. M., Harborth, J., Lendeckel, W., Yalcin, A., Weber, K. and Tuschl, T. (2001) Duplexes of 21-nucleotide RNAs mediate RNA interference in cultured mammalian cells. Nature, 411, 494-498.
9. Paddison, P. J., Caudy, A. A., Bernstein, E., Hannon, G. J. and Conklin, D. S. (2002) Short hairpin RNAs (shRNAs) induce sequence-specific silencing in mammalian cells. Genes Dev., 16, 948-958.
10. Donze, O. and Picard, D. (2002) RNA interference in mammalian cells using siRNAs synthesized with T7 RNA polymerase. Nucleic Acids Res., 30, e46.
11. Brummelkamp, T. R., Bernards, R. and Agami, R. (2002) A system for stable expression of short interfering RNAs in mammalian cells. Science, 296, 550-553.
12. McManus, M. T., Petersen, C. P., Haines, B. B., Chen, J. and Sharp, P. A. (2002) Gene silencing using micro-RNA designed hairpins. RNA, 8, 842-850.
13. Paddison, P. J., Caudy, A. A. and Hannon, G. J. (2002) Stable suppression of gene expression by RNAi in mammalian cells. Proc. Natl. Acad. Sci. USA, 99, 1443-1448.
14. Paul, C. P., Good, P. D., Winer, I. and Engelke, D. R. (2002) Effective expression of small interfering RNA in human cells. Nat. Biotechnol., 20, 505-508.
16. Sui, Q., Soohoo, C., Affar, e. B., Gay, F., Shi, Y., Forrester, W. C. and Shi, Y. (2002) A DNA vector-based RNAi technology to suppress gene expression in mammalian cells. Proc. Natl. Acad. Sci. USA, 99, 5515-6520.
16. Tuschl, T. (2002) Expanding small RNA interference. Nat Biotechnol, 20, 446-448.
17. Yu, J. Y., DeRuiter, S. L. and Turner, D. L. (2002) RNA interference by expression of short-interfering RNAs and hairpin RNAs in mammalian cells. Proc. Natl. Acad. Sci. USA, 99, 6047-6052.
18. Lee, N. S., Dohjima, T., Bauer, G., Li, H., Li, M. J., Ehsani, A., Salvaterra, P. and Rossi, J. (2002). Expression of small interfering RNAs targeted against HIV-1 rev transcripts in human cells. Nat. Biotechnol., 20, 500-505.
19. Xia, H., Mao, Q., Paulson, H. L. and Davidson, B. L. (2002) siRNA-mediated gene silencing in vitro and in vivo. Nat. Biotechnol., 20, 1006-1010.
20. Denti, M. A., Rosa, A., Sthandler, O., De Angelis, F. G. and Bozzoni, I. (2004) A new vector, based on the PolII promoter of the U1 snRNA gene, for the expression of siRNAs in mammalian cells. Mol. Ther., 10, 191-199.
21. Schramm, L. and Hernandez, N. (2002) Recruitment of RNA polymerase III to its target promoters. Genes Dev., 16, 2593-2620.
22. Kawasaki, H. and Taira, K. (2003) Short hairpin type of dsRNAs that are controlled by tRNA^{Val} promoter significantly induce RNAi-mediated gene silencing in the cytoplasm of human cells. Nucleic Acids Res., 31, 700-707.
23. Boden, D., Pusch, O., Lee, F., Tucker, L., Shank, P. R. and Ramratnam, B. (2003) Promoter choice affects the potency of HIV-1 specific RNA interference. Nucleic Acids Res., 31, 5033-5038.
24. Pfeffer, S., Zavolan, M., Grasser, F. A., Chien, M., Russo, J. J., Ju, J., John, B., Enright, A. J., Marks, D., Sander, C. et al. (2004) Identification of virus-encoded microRNAs. Science, 304, 734-736.
25. Thimmappaya, B., Weinberger, C., Schneider, R. J. and Shenk, T. (1982) Adenovirus VAI RNA is required for efficient translation of viral mRNAs at late times after infection. Cell, 31, 543-551.
26. Howe, J. G. and Shu, M. D. (1989) Epstein-Barr virus small RNA (EBER) genes; unique transcription units that combine RNA polymerase II and III promoter elements. Cell, 57, 825-834.
27. Howe, J. G. and Shu, M. D. (1993) Upstream basal promoter element important for exclusive RNA polymerase III transcription of the EBER 2 gene. Mol. Cell Biol., 13, 2665-2665.
28. Wensing, B., Stuhler, A., Jenkins, P., Hollyoake, M., Karstegi, C. E. and Farrell, P. J. (2001) Variant chromatin structure of the oriP region of Epstein-Barr virus and regulation of EBER1 expression by upstream sequences and oriP. J. Virol., 75, 6235-6241.
29. Zhou, Y., Ching, Y. P., Kok, K. H., Kung, H. F. and Jin, D. Y. (2002) Post-transcriptional suppression of gene expression in Xenopus embryos by small interfering RNA. Nucleic Acids Res., 30, 1664-1669.
30. Hannon, G. J. and Rossi, J. J. (2004) Unlocking the potential of the human genome with RNA interference. Nature, 431, 371-378.
31. Dumpelmann, E., Mittendorf, H. and Benecke, B. J. (2003) Efficient transcription of the EBER2 gene depends on the structural integrity of the RNA. RNA, 9, 432-442.
32. Howe, J. G. and Steitz, J. A. (1986) Localization of Epstein-Barr virus-encoded small RNAs by in situ hybridization. Proc. Natl. Acad. Sci. USA, 83, 9006-9010.
33. Meister, G. and Tuschl, T. (2004) Mechanisms of gene silencing by double-stranded RNA. Nature, 431, 343-3493
34. Robb, G. B., Brown, K. M., Khurana, J. and Rana, T. M. (2005) Specific and potent RNAi in the nucleus of human cells. Nat. Struct. Mol. Biol., 12, 133-137.
35. Morris, K. V., Chan, S. W., Jarobsen, S. E. and Looney, D. J. (2004) Small interfering RNA-induced transcriptional gene silencing In human cells. Science, 305, 1289-1292.
36. Kawasaki, H. and Taira, K. (2004) Induction of DNA methylation and gene silencing by short interfering RNAs in human cells. Nature, 431, 211-217.
37. Stevenson, M. (2004) Therapeutic potential of RNA interference. New Engl. J. Med., 351, 1772-1777.

We claim:

1. An expression cassette for a vector system for expression and delivery of desired siRNAs into a host cell, which comprises: a promoter of Epstein-Barr virus-encoded small RNA 1 (EBER1) or an Epstein-Barr virus-encoded small RNA 2 (EBER2) promoter, operably linked to a nucleic acid sequence.

2. The expression cassette of claim 1, wherein the promoter comprises an Epstein-Barr virus-encoded small RNA 2 (EBER2).

3. The expression cassette of claim 1, wherein the siRNA comprises first and second regions, and wherein at least a portion of the first region is complementary to the second region so that a double stranded RNA comprising about 18 to about 25 nucleotides is formed.

4. The expression cassette of claim 2, wherein the siRNA comprises first and second regions, and wherein at least a portion of the first region is complementary to the second region so that a double stranded RNA comprising about 18 to about 25 nucleotides is formed.

5. The expression cassette of claim 3, wherein the siRNA is a small hairpin RNA (shRNA).

6. The expression cassette of claim 4, wherein the siRNA is an shRNA.

7. The vector system of claim 3, wherein at least a portion of the siRNA is complementary to a target RNA, and wherein the portion is about 15 to about 19 nucleotides in length.

8. The vector system of claim 4, wherein at least a portion of the siRNA is complementary to a target RNA, and wherein the portion is about 15 to about 19 nucleotides in length.

9. A method for inhibiting the function of a target RNA, which comprises transfecting a cell with the vector system comprising the expression cassette of claim 3.

10. A method for Inhibiting the function of a target RNA, which comprises transfecting a cell with a vector system comprising the expression cassette of claim 4.

11. The method of claim 9, wherein the cell is a mammalian cell.

12. The method of claim 9, wherein the cell is a human cell.

13. The method of claim 9, wherein the cell is in an organism.

14. The method of claim 9, wherein the cell is transplanted into an organism.

15. The method of claim 10, wherein the cell is a mammalian cell.

16. The method of claim 10, wherein the cell is a human cell.

17. The method of claim 10, wherein the cell is in an organism.

18. The method of claim 10, wherein the cell is transplanted into an organism.


SEQUENCE LISTING (20 sequences)

SEQ ID NO: 1 - DNA, 22 bp, Artificial Sequence
  EBER Promoter
ggaaatgagggttagcataggc

SEQ ID NO: 2 - DNA, 110 bp, Artificial Sequence
  EBER Promoter
aaaaaaatcaggtggctcccgctgaattggaatcccaagcttcggattccaactcagcgagagccacccgatctcgagatcgattctagaagacaaccacagacaccgtc

SEQ ID NO: 3 - DNA, 22 bp, Artificial Sequence
  EBER Promoter
gcttaacgttgcatcccagaag

SEQ ID NO: 4 - DNA, 112 bp, Artificial Sequence
  EBER Promoter
aaaaaaatcaggtggctcccgctgaattggaatcccaagcttcggattccaactcagcgagagccacccgatctcgagatcgattctagactgacttgcaaatgctctaggc

SEQ ID NO: 5 - DNA, 22 bp, Artificial Sequence
  EBER Promoter
ggaaatgagggttagcataggc

SEQ ID NO: 6 - DNA, 110 bp, Artificial Sequence
  EBER Promoter
aaaaaagttaggaattataatgcttatctacgtgccaagcttcgcacatagataagcactataatccctaacctcgagatcgattctagaagacaaccacagacaccgtc

SEQ ID NO: 7 - DNA, 22 bp, Artificial Sequence
  EBER Promoter
gcttaacgttgcatcccagaag

SEQ ID NO: 8 - DNA, 112 bp, Artificial Sequence
  EBER Promoter
aaaaaagttaggaattataatgcttatctacgtgccaagcttcgcacatagataagcactataatccctaacctcgagatcgattctagactgacttgcaaatgctctaggc

SEQ ID NO: 9 - DNA, 23 bp, Artificial Sequence
  Human tRNA Promoter
ttgatcccgaaagatgtccagcg

SEQ ID NO: 10 - DNA, 109 bp, Artificial Sequence
  Human Promoter
aaaaaagttaggaattataatgcttatctacgtgccaagcttcgcacatagataagcactataatccctaacctcgagatcgattctagatttccgcccggtttcgaac

SEQ ID NO: 11 - DNA, 18 bp, Artificial Sequence
  Human Promoter
atttaggtgacactatag

SEQ ID NO: 12 - DNA, 110 bp, Artificial Sequence
  Human Promoter
aaaaaagttaggaattataatgcttatctacgtgccaagcttcgcacatagataagcactataatccctaacctcgagatcgattctagacggtgtttcgtcctttccac

SEQ ID NO: 13 - DNA, 18 bp, Artificial Sequence
  Human Promoter
atttaggtgacactatag

SEQ ID NO: 14 - DNA, 130 bp, Artificial Sequence
  Human Promoter
aaaaaagttaggaattataatgcttatctacgtgccaagcttcgcacatagataagcactataatccctaacctcgagatcgattctagatagtatatgtgctgccgaagcgagcacggtgtttcgtcct

SEQ ID NO: 15 - DNA, 20 bp, Artificial Sequence
  Human Promoter
taatacgactcactataggg

SEQ ID NO: 16 - DNA, 128 bp, Artificial Sequence
  Human Promoter
aaaaaagttaggaattataatgcttatctacgtgccaagcttcgcacatagataagcactataatccctaactctagactcgagatcgatgggaaagagtggtctcatacagaacttataagattccc

SEQ ID NO: 17 - DNA, 19 bp, Artificial Sequence
  PCR Primer
acgctgaaagtgtagtaga

SEQ ID NO: 18 - DNA, 16 bp, Artificial Sequence
  PCR Primer
agaatcctgggtccga

SEQ ID NO: 19 - DNA, 18 bp, Artificial Sequence
  PCR Primer
tctatccgctggaagatg

SEQ ID NO: 20 - DNA, 18 bp, Artificial Sequence
  PCR Primer
actgttgagcaattcacg